(19) United States
(12) Patent Application Publication
(10) Pub. No.: US 2020/0197671 A1
(43) Pub. Date: Jun. 25, 2020

(54) BALLOON CATHETER WITH WORKING TUBE

(71) Applicant: BIOTRONIK AG, Buelach (CH)

(72) Inventors: Fabian Risch, Doerflingen (CH); Tobias Schäfer, Blumberg-Fuetzen (DE)

(21) Appl. No.: 16/646,086

(22) PCT Filed: Sep. 17, 2018
(86) PCT No.: PCT/EP2018/075042

§ 371 Date: Mar. 10, 2020

(30) Foreign Application Priority Data
Oct. 18, 2017 (EP) 17196972.8

(51) Int. Cl. A61M 25/10

(52) U.S. Cl. CPC A61M 25/1006; A61M 25/1011; A61M 2025/109; A61M 2025/1086; A61M 2025/1072

(57) ABSTRACT

A balloon catheter has a catheter shaft, an expandable balloon and a working tube. The working tube is inflatable, has a proximally open end and distally closed end and is disposed on a periphery of the balloon.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2018/075042, which was filed Sep. 17, 2018, which application claimed priority from European Application EP17196972.8, which was filed Oct. 18, 2017.

### FIELD OF THE INVENTION

The invention relates to a balloon catheter having at least a catheter shaft and an expandable balloon. The balloon catheter of the invention is useful for working on vascular deposits.

### BACKGROUND

Balloon catheters and balloon catheter-stent devices are used in a clinical setting for the treatment of vasoconstrictions (stenoses) of blood vessels. Treatment by expansion (dilatation) of a balloon catheter is often supplemented by a stabilisation of the expanded vessel portion with a support element, that is to say the stent expanded by the balloon and left behind at the location of the stenosis following the deflation of the balloon and the withdrawal of the balloon catheter (stenting). Reference is made, purely by way of example, to DE 102004059523 A1, which describes a new stage of development of these devices.

Often, at least partial removal of deposits on the vessel wall is desirable in addition to the expansion and stabilisation of the stenosis portion. For this purpose, balloon catheters having what are known as cutting balloons with cutting blades on the balloon surface and more recently also stents having scoring elements for cutting vascular deposits have been developed and are also used already in a clinical setting.

Document WO 2009/046206 A1 presents a balloon catheter with attached scoring elements. The scoring elements are helically extending metal wires or comparable plastic elements attached to the balloon of the balloon catheter. Document U.S. Pat. No. 8,348,987 B2 presents a balloon catheter-stent device with scoring elements attached to the stent structure.

It has been found that both solutions have significant disadvantages.

Cutting balloons are relatively rigid on account of the attached cutting elements and can only be introduced with difficulty into complex lesions, and there is a significant risk of perforation. In addition, the minimum (deflated) balloon diameter is larger than in comparable balloons without cutting elements. Lastly, cutting balloons put up a relatively high resistance as the device is advanced through the vessel system, which hinders precise handling.

In stents with scoring elements there is the risk, inter alia, that the scoring elements, which also protrude in the insertion state, might cause tissue damage. On the other hand, the effects actually attained at the intended site of insertion have not proven to be satisfactory in clinical studies.

### SUMMARY OF THE INVENTION

A balloon catheter having a catheter shaft, an expandable balloon and a working tube, wherein the working tube is inflatable, has a proximally open end and distally closed end and is disposed on a periphery of the balloon

A balloon catheter having a catheter shaft, an expandable balloon and a working tube disposed on an outer surface of the balloon, the working tube having a first size when the balloon is deflated, wherein the working tube has a fluid connection to be inflated to a second larger size when the balloon is inflated.

### BRIEF DESCRIPTION OF THE DRAWINGS

Advantages and expedient features of the invention will also become clear from the following description of an exemplary embodiment with reference to the drawings, in which:

FIG. 1 shows a schematic perspective view of a balloon catheter in accordance with a first embodiment of the invention,

FIG. 2 shows a schematic perspective view of a balloon catheter in accordance with a further embodiment of the invention,

FIG. 3 shows a schematic perspective view of a balloon catheter in accordance with a further embodiment of the invention,

FIG. 4 shows a schematic longitudinal sectional view of the main structure of a balloon catheter,

FIG. 5 shows a schematic longitudinal sectional view of a second main structure of a balloon catheter,

FIG. 6 shows a schematic longitudinal sectional view of a balloon catheter in accordance with a further embodiment of the invention.

### DETAILED DESCRIPTION OF THE PREFERRED EMBODIMENTS

The invention provides balloon catheter with working tubes which in the starting sate with deflated balloon, that is to say during the insertion of the balloon catheter, have a different physical characteristic than in the end state with inflated balloon, in which they are intended to be suitable for working on a stenosis. In particular, in the starting state they have a low rigidity and protrude to the smallest possible extent beyond the peripheral surface of the (as yet uninflated) balloon, whereas in the end state they protrude beyond the balloons surface to an extent sufficient for working and are inflated to be sufficiently rigid.

The invention also includes the concept of bringing about this “state transition” in a manner similar to that implemented in the case of the catheter balloon itself, specifically by filling with a suitable liquid. Lastly, the invention includes the concept that the working means have at least one additional tube with a closed end, which tube is attached with a suitable profile to the balloon of the catheter and will be referred to hereinafter as an working tube.

Within the scope of this application, a balloon catheter is understood to mean a catheter which is suitable for being introduced into a bodily vessel or lumen of a patient, in particular into a lumen of the bloodstream. A balloon catheter of this kind includes at least one catheter shaft and an expandable balloon, referred to as a balloon for short. The expansion (inflation) of the balloon is implemented by acting on said balloon with a fluid via a lumen of the catheter shaft.

In expedient embodiments of the invention a plurality of working tubes of this kind, in particular 3 to 5 working tubes, is provided. Improved working effects can be achieved hereby compared with an individual working tube.

In particular, the working tube or the working tubes lies/lie helically on the balloon, wherein it/they describes/describe in particular at least a half turn about the longitudinal axis of the balloon between the proximal balloon neck and the distal balloon neck. The angular range of a helical course between proximal and distal balloon neck of the working tube or the working tubes will be determined by a person skilled in the art in accordance with the specific configuration, in particular in respect of the length of the balloon, the number of attached tubes, and requirements of the specific application. In particular if a plurality of working tubes are provided, it is also possible in principle to lay said working tubes parallel to the longitudinal axis of the balloon.

In an embodiment of the invention currently considered to be expedient, the working tube or each working tube is welded or glued at least over the majority of its length to the balloon. Alternatively, it is possible that the working tube is fixedly connected, in particular welded or adhesively bonded, to the catheter shaft substantially only at the proximal balloon neck. In the latter variant it would appear to be advantageous if the working tube or each working tube is also welded or glued therebetween at specific points to the balloon so as to prevent undesirable tangential and/or axial displacements of the tube or the tubes relative to the balloon and associated deteriorations of the working effect.

In a further embodiment the catheter shaft includes an inner shaft and an outer shaft, and the open proximal end of the working tube or each working tube is connected fluidically to the lumen of the outer shaft. The working tube or the working tubes is/are pressurised at this moment, that is to say simultaneously with the catheter balloon, which makes the structure of the catheter as a whole and also handling thereof particularly simple. In another catheter construction known per se, the catheter shaft has a first lumen for a guide wire and a second lumen for introducing a fluid for expanding the balloon, and the open proximal end of the working tube or each working tube is fluidically connected to the second lumen.

Alternatively, it is provided that the open proximal end of the working tube or each working tube opens out into a separate proximal fluid connection of the balloon catheter. This offers the possibility of controlling the characteristic of the working means independently of the inflation of the balloon, but requires a separate fluid line extending as far as the proximal catheter end or an additional lumen in the catheter body and a separate connection of the catheter.

Here, it is possible on the one hand that the open proximal end of the working tube or each working tube is connected via an additional lumen in the catheter shaft to the dedicated fluid connection. Alternatively, as a modification of known catheter configurations, the proximal end of the working tube or each outer tube can be connected to the separate fluid connection via a working fluid tube laid externally on the catheter shaft.

In a further embodiment of the invention the balloon catheter has a protective sleeve displaceable along the catheter shaft. This protective sleeve is advanced as far as the distal balloon neck before and during insertion of the catheter and covers the working tube or the working tubes, and is withdrawn as far as at least the proximal balloon neck after the insertion, so as to expose the working tube or the working tubes for implementation of its/their working purpose. An additional actuation element is required for the protective sleeve, and the handling of the catheter of this kind is slightly more complex.

In expedient embodiments the working tube or each working tube includes a material from the group of polyamides, polyimides, polyesters, polyethers, polyolefins and copolymers thereof. Materials which are compatible with the material of the catheter balloon such that light welding or adhesive bonding is possible are particularly preferred.

In further embodiments of the invention the diameter of the working tube or each working tube lies in the range between 2% and 8%, preferably between 2% and 4%, of the diameter of the balloon. The specific determination of the working tube geometry will be made by a person skilled in the art as a matter of routine, in particular under consideration of the structure of the balloon catheter per se and the specific situation of application.

In further embodiments it is provided that the working tube or at least part of the working tubes includes a cutting or sawing edge for abrasive working on vascular deposits. The cutting or sawing edge(s) can be adhesively bonded or welded to the working tube proximally, distally and/or over the entire length. Alternatively, the cutting or sawing edge(s) can also be produced directly with the forming of the working tube. Materials that are suitable for the cutting or sawing edge are in particular metals (for example stainless steel) or polymers, such as polyamides, polyimides, polyesters, polyethers, polyolefins and copolymers thereof. The diameter of an working tube (or maximum radial extent thereof) is advantageously between 4% and 8% of the balloon diameter. This is advantageous in particular for applications in which the balloon catheter should not only break through calcareous deposits, but should also separate and/or remove them in layers. Cutting or sawing edges can slightly hamper the handling of the balloon catheter during the insertion process; this disadvantage, however, is outweighed in certain application situations by the advantages of an embodiment of this kind.

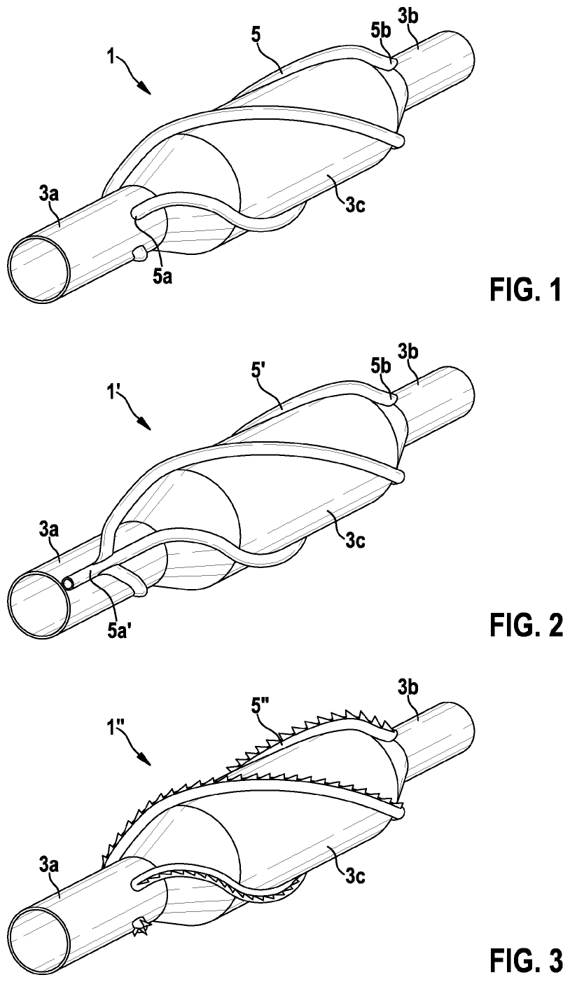

FIG. 1 shows a sketch of a balloon catheter 1 which includes an outer shaft 3a, an inner shaft 3b, and a balloon 3c (shown in the inflated state in the drawing), and also three working tubes 5 laid on the balloon 3c. The working tubes 5 laid helically at equal angular distances over the peripheral surface of the balloon 3c each have a proximal open end. They are connected there fluidically at mouth points 5a to the inner lumen of the catheter shaft 3a, that is to say are subjected to fluid pressure simultaneously with the inflation of the balloon 3c and in the same way as the balloon. At their distal ends 5b, the working tubes 5 are closed in a fluid-tight manner. The working tubes can be welded, adhesively bonded or otherwise fixedly connected over their entire surface or also only at specific points, for example at the neck portions of the balloon 3c, to the balloon 3c and/or the adjoining shaft portions.

FIG. 2 shows a modification of the structure shown in FIG. 1, wherein the same reference numerals are provided for matching or functionally similar parts, and those parts will not be described further here. The main difference of the balloon catheter 1′ from the balloon catheter 1 according to FIG. 2 lies in a modified embodiment of the working tubes 5′ at the proximal ends thereof. Here, these working tubes specifically do not open out into the catheter shaft 3a, but instead are connected to one another via a branch piece 5a′ and are connected to a separate fluid line (not shown) leading to the proximal catheter end. In this embodiment the working tubes 5′ are placed under fluid pressure via the separate fluid line, independently of the inflation of the balloon 3c.

FIG. 3 shows a further balloon catheter 1″, which is derived from the balloon catheter 1 according to FIG. 1 by way of a modification. The main difference here lies in the fact that a sawtooth structure is formed on the working tubes 5″and enables abrasive working on vascular deposits at the site of use of the balloon catheter.

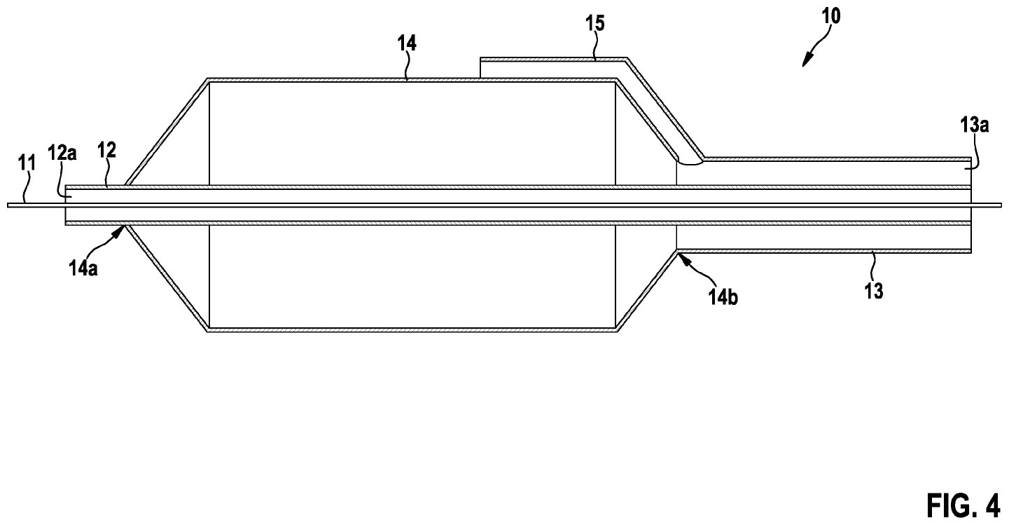

FIG. 4 schematically shows, in the form of a longitudinal sectional view, a first variant of the inner structure of a balloon catheter 10 according to the invention with a substantially coaxial structure. The catheter shaft (not referenced separately here) includes an inner shaft 12, in which a guide wire 11 is received, and an outer shaft 13. A balloon 14 is welded to the inner shaft 12 at a distal balloon neck 14a and to the outer shaft 13 at a proximal balloon neck 14b. The balloon 14 is shown here in the expanded state. In order to produce this state, a lumen 13a for feeding a fluid into the balloon 14 is provided in the outer shaft 13, whereas an inner shaft lumen 12a coaxial with the lumen 13a receives the guide wire 11 longitudinally displaceably in the inner shaft 12. An working tube 15 as working means of the balloon catheter 10 is fluidically connected at the proximal balloon neck 14b to the outer shaft lumen 13a. The working tube 15 is thus inflated simultaneously with the balloon 14.

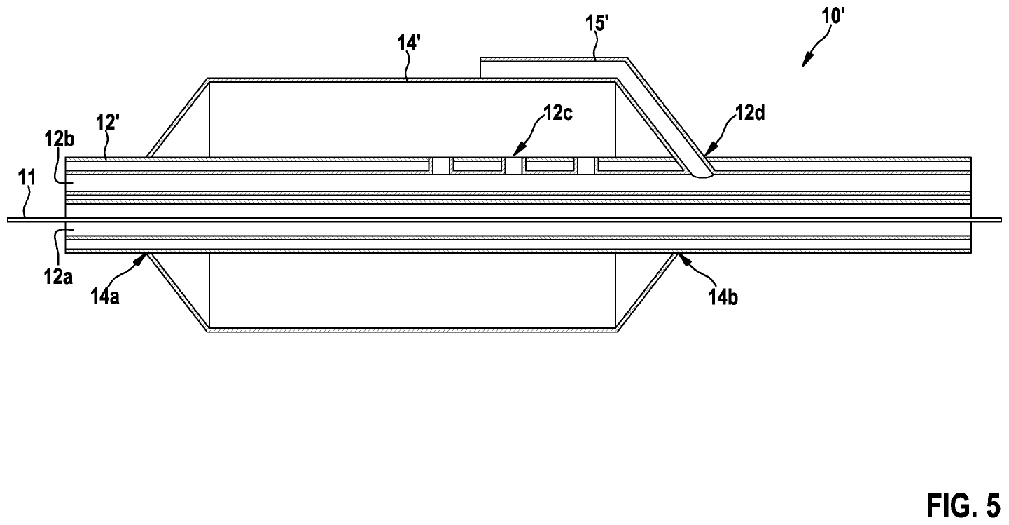

FIG. 5 shows a further balloon catheter 10′ with a different structure, wherein like or functionally comparable parts are denoted by the same numbers as in FIG. 4 and will not be described here again. In contrast to the balloon catheter 10 according to FIG. 4, the balloon catheter 10′ has a one-piece catheter shaft 12′, in which two lumens 12a (for the guide wire 11) and 12b (as fluid channel) are incorporated parallel to one another. The catheter shaft 12′ is welded to the balloon 14′ both at the distal balloon neck 14a and at the proximal balloon neck 14b. The balloon 14′ is filled via passages 12c from the fluid channel 12b into the interior of the balloon 14′ with a fluid fed from the proximal catheter and is expanded hereby. A further passage 12d connects the fluid channel 12b to the working tube 15′. Fluid in the lumen 12b thus again places the working tube under fluid pressure simultaneously with the balloon 14′ and expands said tube. Alternatively to the shown configuration, the working tube 15′ can also run externally on the catheter shaft 12′ to the proximal catheter end.

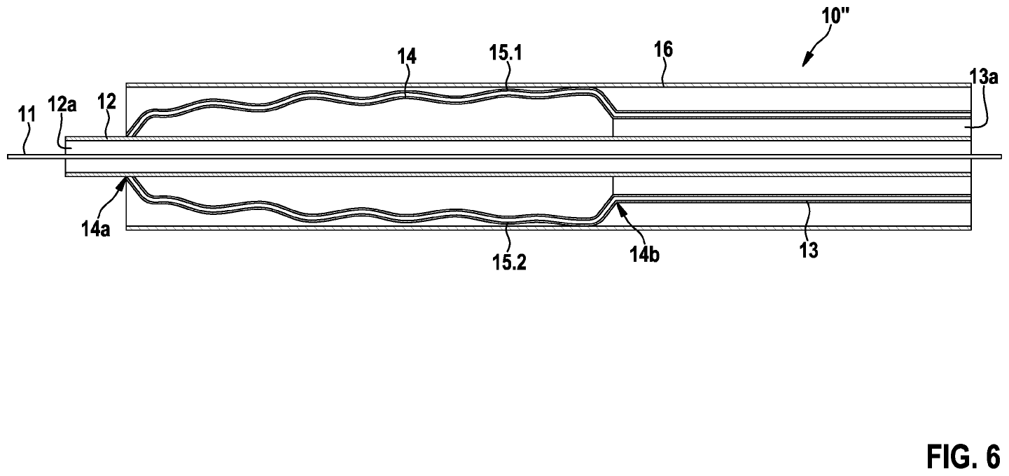

A further alternative to the configurations shown in FIG. 4 and FIG. 5 lies in that an additional lumen is provided within the outer catheter in the arrangement according to FIG. 4 or within the catheter shaft in the arrangement according to FIG. 5, which additional lumen is used exclusively to supply fluid to the working tube or the working tubes. This variant is shown schematically in FIG. 6, more specifically together with a further modification of the configuration according to FIG. 4. This lies in that a protective sleeve 16 is associated with the actual balloon catheter C2′. This protective sleeve covers the working tubes 15.1 and 15.2 (and any further working tubes; not shown here) in the insertion state and is withdrawn proximally after insertion, when the balloon catheter is placed in operation with its working tubes serving as a tool to remove vascular deposits.

Otherwise, the invention can also be embodied in a large number of modifications of the examples shown here and aspects of the invention described further above.

1-15. (canceled)

16. A balloon catheter comprising a catheter shaft, an expandable balloon and a working tube, wherein the working tube is inflatable, comprises a proximally open end and distally closed end and is disposed on a periphery of the balloon.

17. The balloon catheter according to claim 1, comprising a plurality of inflatable working tubes disposed on the periphery of the balloon.

18. The balloon catheter according to claim 17, comprising three to five working tubes.

19. The balloon catheter according to claim 16, wherein the working tube is arranged helically on the balloon.

20. The balloon catheter according to claim 19, wherein the working tube forms at least one half turn about the longitudinal axis of the balloon between a proximal balloon neck and a distal balloon neck.

21. The balloon catheter according to claim 16, wherein the working tube is welded or glued at least over the majority of its length to the balloon.

22. The balloon catheter according to claim 16, wherein the working tube is welded or glued to the catheter shaft substantially only at a proximal balloon neck and at a distal balloon neck.

23. The balloon catheter according to claim 16, wherein the working tube is welded or glued at a plurality of positions between a proximal and a distal balloon neck.

24. The balloon catheter according to claim 16, wherein the catheter shaft comprises an inner shaft and an outer shaft, and the open proximal end of the working tube is fluidically connected to a lumen of the outer catheter.

25. The balloon catheter according to claim 16, wherein the catheter shaft comprises a first lumen for a guide wire and a second lumen for introducing a fluid for expanding the balloon, and the open proximal end of the working tube is fluidically connected to the second lumen.

26. The balloon catheter according to claim 16, comprising a dedicated proximal fluid connection to the open proximal end of the working tube.

27. The balloon catheter according to claim 26, comprising a plurality of inflatable working tubes connected to the dedicated fluid connection via an additional lumen in the catheter shaft.

28. The balloon catheter according to claim 26, wherein dedicated proximal fluid connection comprises a fluid tube disposed on the catheter shaft.

29. The balloon catheter according to claim 16, comprising a protective sleeve displaceable along the catheter shaft.

30. The balloon catheter according to claim 16, wherein the working tube comprises a material selected from the group consisting of polyamides, polyimides, polyesters, polyethers, polyolefins and copolymers thereof.

31. The balloon catheter according to claim 16, wherein a diameter of the working tube is in the range between 2% and 8% of the diameter of the balloon.

32. The balloon catheter according to claim 31, wherein the diameter of the working tube is between 2% and 4% of the diameter of the balloon,

33. The balloon catheter according to claim 16, comprising a cutting or sawing edge on the working tube.

34. A balloon catheter comprising a catheter shaft, an expandable balloon and a working tube disposed on an outer surface of the balloon, the working tube having a first size when the balloon is deflated, wherein the working tube has a fluid connection to be inflated to a second larger size when the balloon is inflated.
